(19) United States
(12) Patent Application Publication
(10) Pub. No.: US 2007/0098665 A1
(43) Pub. Date: May 3, 2007

(54) Effects of skin surface temperature on the epidermal permeability barrier homeostasis

(75) Inventors: Mitsuhiro Denda, Yokohama-shi (JP); Kaori Inoue, Yokohama-shi (JP)

Correspondence Address: SNIDER & ASSOCIATES, P. O. BOX 27613, WASHINGTON, DC, 20038-7613, US

(73) Assignee: Shiseido Company, Ltd., Tokyo (JP)

(21) Appl. No.: 11/585,229

(22) Filed: Oct. 24, 2006

Related U.S. Application Data
(60) Provisional application No. 60/729,208, filed on Oct. 24, 2005.

(51) Int. Cl. A61K 8/64; A61K 8/65

(52) U.S. Cl. 424/70.14

(57) ABSTRACT

A method for accelerating barrier recovery of skin by activating the TRPV4 and/or by blocking the TRPV1 on epidermal cell is provided.

### FIELD OF THE INVENTION

The present invention relates to a method for accelerating barrier recovery of skin.

### PRIOR ART

One of the most important roles of the skin for terrestrial mammals is to generate a water-impermeable barrier against excess transcutaneous water loss. A decline in barrier function often parallels increased severity of clinical symptomatology (Elias and Feingold 2001). When the stratum corneum barrier is damaged, a series of homeostatic processes in the barrier function is immediately accelerated, and the barrier recovers to its original level (Elias and Feingold 2001).

Previously, Grubauer et al. demonstrated that occlusion with water impermeable membrane immediately after barrier disruption blocked the barrier recovery, while occlusion with water permeable membrane did not perturb the barrier recovery (Grubauer et al. 1989). These results suggest that there might be a sensor of water flux from skin surface and the monitoring system regulate the barrier homeostasis.

Recently, TRP receptor family has been reported as a sensor of temperature or other physical or chemical factors. Moreover, existences of TRPV1, TRPV3 and TRPV4 in epidermal keratinocytes were reported.

### DISCLOSURE OF THE INVENTION

Recently, a series of receptors called TRP family are reported as sensor of temperature. Moreover, some of them are expressed in the epidermal keratinocytes. To evaluate the influence of these receptors on the epidermal permeability barrier homeostasis, we applied different temperature on both hairless mice and human skin immediately after tape stripping. When we applied the heat between 36° C. to 40° C., the barrier recovery was accelerated on both human and mice skin. When we applied the heat below 34° C. or at 42° C., the barrier recovery was delayed. When we topically applied 4 alpha-PDD, an activator of TRPV4, which is also activated by the heat over 35° C., on the hairless mice skin after tape stripping, the barrier recovery was accelerated while when we applied Rutenium-Red, a blocker of TRPV4, the barrier recovery was delayed. On the other hand, when we topically applied capsaicin, an activator of TRPV1, which is also activated by the heat over 42-43° C., the barrier recovery was delayed while application of capsazepin, an antagonist of TRPV1, the delay was blocked. These results suggest that both TRPV1 and TRPV4 play an important role on skin permeability barrier homeostasis. Thus, the results of the present study suggest new insight on the epidermal physiology regarding environmental temperature.

Accordingly, in the first aspect, the present invention provides a method for accelerating barrier recovery of skin by activating the TRPV4 on epidermal cell.

In the second aspect, the present invention provides a method for accelerating barrier recovery of skin by blocking the TRPV1 on epidermal cell.

Further, the present invention provides a method for accelerating barrier recovery of skin by activating the TRPV4 on epidermal cell, while blocking the TRPV1 on epidermal cell.

### BRIEF DESCRIPTIONS OF DRAWINGS

FIG. 1 show the effects of application of different temperature immediately after tape-stripping on hairless mice skin. Immediately after application of heat 36-40° C. for 1 hour, the barrier recovery was accelerated, while application of 32, 34 and 42° C. delayed the barrier recovery. The tendencies were observed within 3-6 hours after barrier disruption.

FIG. 2 show the effects of application of different temperature immediately after tape-stripping on human skin. Same tendencies were observed as the results of hairless mice experiments on FIG. 1. Immediately after application of heat 36-40° C. for 1 hour, the barrier recovery was accelerated, while application of 34 and 42° C. delayed the barrier recovery. The tendencies were observed within 3-6 hours after barrier disruption.

FIG. 3 show the effects of topical application of activators and inhibitors of each TRP receptors. Topical application of capsaicin, a TRPV1 activator, on the hairless mice skin after tape-stripping delayed the barrier recovery, while application of capsazepine, a TRPV1 blocker, accelerated the barrier repair. On the other hand, topical application of 4 alpha-PDD, a TRPV4 activator accelerated the barrier recovery, while application of Rutenium-Red delayed the recovery. Application of 2-aminoethoxydiphenyl borate and camphor, both TRPV3 activator, did not affect the barrier recovery rate.

FIG. 4 illustrate that Rutenium-Red and capsazepine blocked the effects of the temperature of skin surface on the barrier recovery rate. Topical application of capsazepine blocked the delay of the barrier recovery by the heat of 42° C. and application of Rutenium-Red blocked the acceleration of the barrier recovery by the heat of 36° C.

### BEST MODE FOR CARRYING OUT THE INVENTION

The present invention is based on the discovery that TRPV1 and TRPV4 play an important role on skin permeability barrier homeostasis.

The method for accelerating barrier recovery of skin comprises the step of activating the TRPV4 on epidermal cell. Activation of the TRPV4 can be accomplished, for example, by applying heat at a temperature of between 36 to 40° C., for an appropriate period, such as a few minutes to a few hours, e.g. 10 minutes to 1 hour, and/or by applying an activator or an agonist of the TRPV4, for example, 4 alpha-PDD (4 alpha-phorbol 12,13-didecanoate) onto the skin.

Further, another method for accelerating barrier recovery of skin comprises the step of blocking the TRPV1 on epidermal cell. Blocking of the TRPV1 can be accomplished, for example, by applying heat at a temperature of between 36 to 40° C., for an appropriate period, such as a few minutes to a few hours, e.g. 10 minutes to 1 hour, and/or by applying a blocker or an antagonist of TRPV1, for example, capsazepin, onto the skin.

### EXAMPLES

Material

All experiments were performed on 7-10-week old male hairless mice (HR-1, Hoshino, Japan). All procedures for measuring of skin barrier function, disrupting the barrier and applying the sample were carried out under anesthesia. All experiments were approved by the Animal Research Committee of the Shiseido Research Center in accordance with the National Research Council Guide (National Research Council 1996). Human skin experiments were carried out on the inner forearm of healthy males who gave their informed consent. 4 alpha-phorbol 12,13-didecanone (4 alpha-PDD) and Rutenium-Red were purchased from Sigma (Sigma, St.Louis, Mich. USA). Capsaicin, capsazepine, and 2-aminoethoxydiphenyl borate (2-APB) were purchased from Tocris (TOCRIS, Bristol, UK). Camphor was purchased Wako (Wako, Osaka, Japan). Capsaicin is an agonist of TRPV1 and capsazepine is an antagonist of TRPV1 (Caterina et al. 1997). Both 2-APB and camphor are activator of TRPV3 (Chung et al. 2003)(Moquich et al. 2005). 4 alpha-PDD is an activator of TRPV4 and Rutenium-Red is a blocker of TRPV4 (Watanabe et al. 2002).

Cutaneous Barrier Function

Permeability barrier function was evaluated by measurement of transepidermal water loss (TEWL) with an electric water analyzer, as described previously (Denda et al. 1998). For barrier recovery experiments, both sides of flank skin were treated with repeated tape stripping procedure until the TEWL reached 7-10 mg per cm² per h, as described previously (Denda et al. 1998). Immediately after barrier disruption, 100 μl of an aqueous solution containing 1 μM of reagent or water alone (control) was applied to the treated area. We did not apply same reagent in both flank. The areas were covered with plastic membranes for 15 min and then the membranes were removed. Two points in one side of flank were measured and 4-8 mice were used to evaluate the effects of each treatment. We always disrupted the barrier within 7:00 AM to 8:00 AM and carried out the following measurements of the barrier repair to avoid the deviation of the repair rate due to the influence of circadian rhythm (Denda et al. 2000). TEWL was then measured over the same sites at 1, 3 and 6 hours after barrier disruption. The barrier recovery results are expressed as percent of recovery, because of variations from day to day in the extent of barrier disruption. In each animal, the percentage of recovery was calculated by the following formula: (TEWL immediately after barrier disruption−TEWL at indicated time point)/(TEWL immediately after barrier disruption−baseline TEWL)×100%. All experiments were performed on 7 to 10-week-old male hairless mice (HR-1, Hoshino, Japan). All procedures of the measurement of skin barrier function, disruption of the barrier and application of test sample were carried out under anesthesia.

Application of Heat

For the application of constant temperature, we used silicone-rubber-coated heater (Sakaguchi Dennetsu, Tokyo, Japan) with Watlow Control System (Sakaguchi Dennetsu, Tokyo, Japan) to keep the temperature of the flexible heater constantly. The size of the heater was 5×10 cm² and immediately after tape stripping, we attached the heater on the treated skin surface directly. One hour after tape stripping, we removed the heater and started to measure the TEWL. Thus, we applied the heat only for one hour at the begging of experiments. On control skin, we did not put anything. During the period of experiments, control skin surface temperature was between 32° C. and 34° C.

Statistics

The results are expressed as the mean ± SD. Statistical differences between two groups were determined by a two-tailed Student's t-test. In the case of more than 2 groups, differences were determined by ANOVA test (Fisher's protected least significant difference).

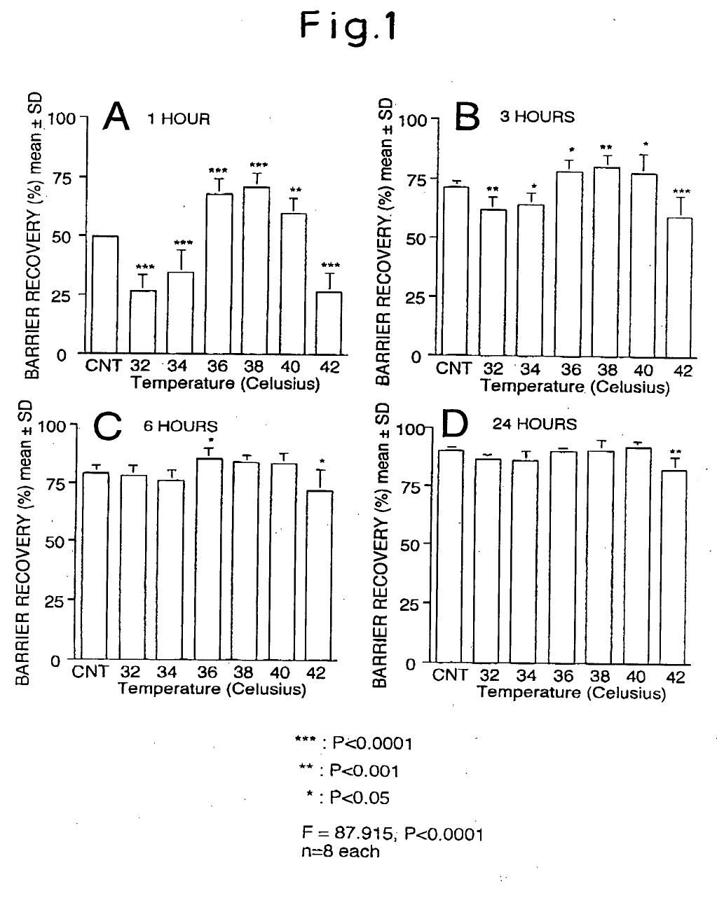

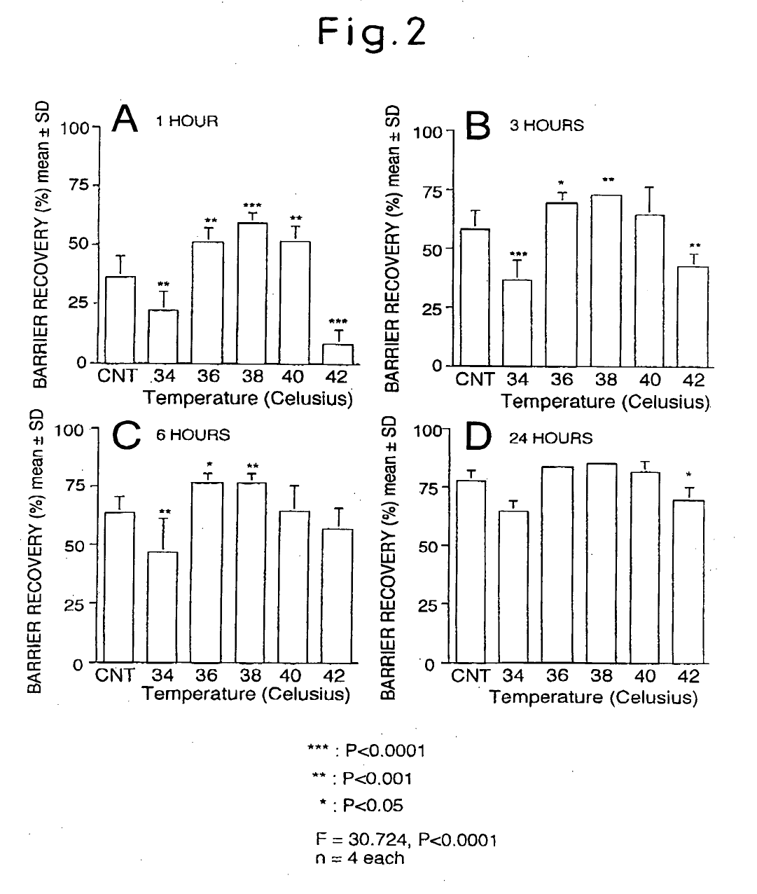

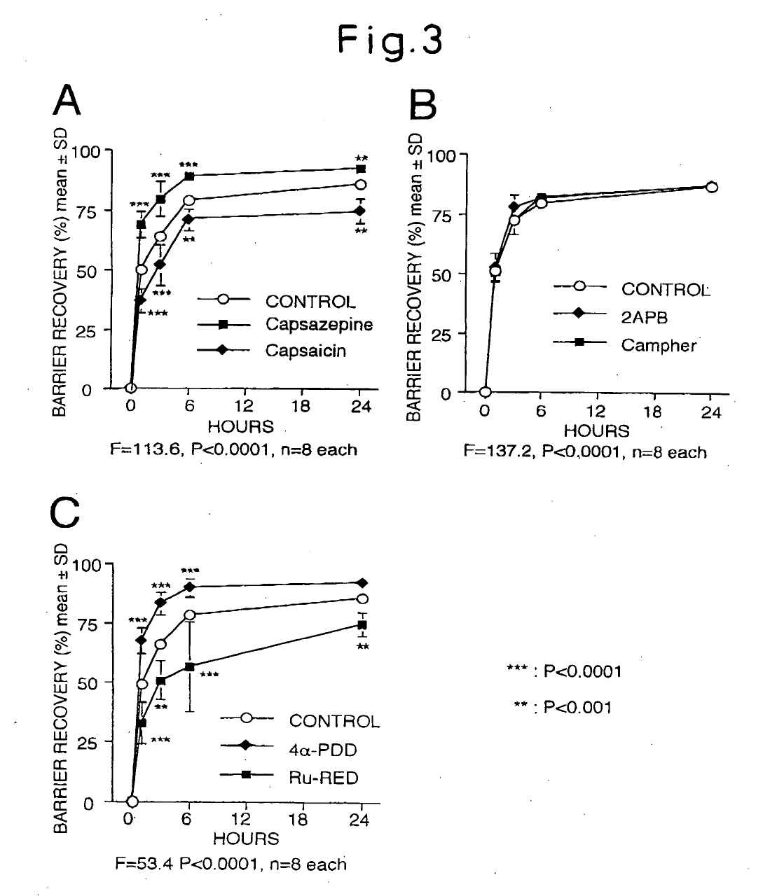

Results When we applied the heat between 36° C. to 40° C., the barrier recovery was accelerated on both mice (FIG. 1) and human skin (FIG. 2). When we applied the heat below 34° C. or at 42° C., the barrier recovery was delayed (FIGS. 1, 2). Topical application of capsaicin, a TRPV1 activator, on the hairless mice skin after tape-stripping delayed the barrier recovery, while application of capsazepine, a TRPV1 blocker, accelerated the barrier repair. On the other hand, topical application of 4 alpha-PDD, a TRPV4 activator, accelerated the barrier recovery, while application of Rutenium-Red delayed the recovery. Application of 2-aminoethoxydiphenyl borate and camphor, both TRPV3 activator, did not affect the barrier recovery rate (FIG. 3).

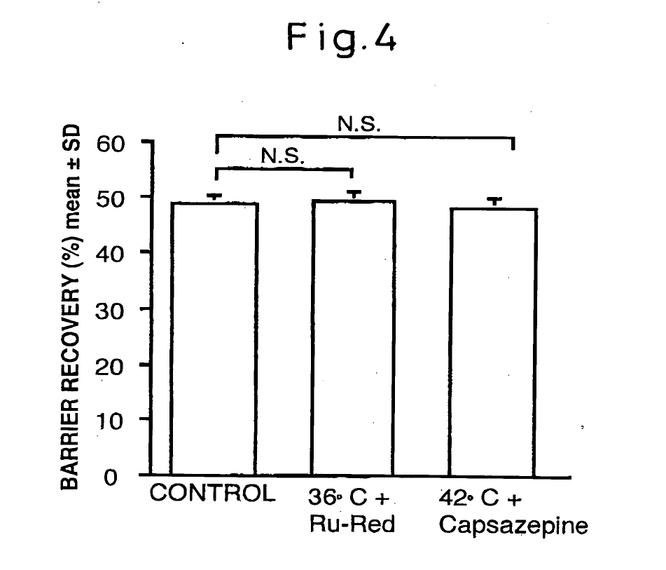

Topical application of capsazepine blocked the delay of the barrier recovery by heat 42° C. and application of Rutenium-red blocked the acceleration of the barrier recovery by heat 36° C. (FIG. 4).

Discussion

In both hairless mice and humans, application of heat between 36° C. to 40° C. accelerated the barrier recovery after the barrier disruption by tape stripping. Both TRPV3 and TRPV4 are activated approximately over 36° C. (Peier et al. 2002)(Chung et al. 2003). But in the present study, topical application of TRPV3 activators did not affect the barrier recovery rate. On the other hand, application of TRPV4 activator, 4 alpha-PDD, accelerated the barrier recovery, while application of TRPV4 blocker, Rutenium-Red delayed it. Moreover, acceleration of the barrier repair by heat 36° C. was blocked by Rutenium-Red. These results suggest that TRPV4, not TRPV3, is associated with the epidermal permeability barrier homeostasis.

When we applied 42° C., on both hairless mice and human skin, the barrier recovery was delayed. TRPV1 is activated approximately 43° C. and topical application of TRPV1 activator, capsaicin delayed the barrier recovery, while application of TRPV1 inhibitor, capsazepine, accelerated the barrier recovery. Moreover, the delay of the barrier recovery by 42° C. was blocked by capsazepine. These results suggest that TRPV1 in the epidermal keratinocytes might be also associated with skin permeability barrier homeostasis.

### REFERENCES

Caterina M J., Schumacher M A., Tominaga M., Rosen T A., Levine J D., Julius D. The capsaicin receptor: A heat-activated ion channel in the pain pathway. Nature 389:816-824

Chung M K., Lee H., Caterina M J. Warm temperatures activate TRPV4 in mouse 308 keratinocytes.

J Biol Chem 2003 278:32037-32046

Chung M K., Lee H., Mizuno A., Suzuki M., Caterina M. 2-aminoethoxydiphenyl borate activates and sensitizes the heat-gated ion channel TRPV3. J Neuroscience 24:5177-5182, 2004

Denda M, Fuziwara S, Inoue K, Denda S, Akamatsu H, Tomitaka A, Matsunaga K, Immunoreactivity of VR1 on epidermal keratinocyte of human skin. Biochem Biophys Res Commun 285:1250-1252 (2001)

Denda M, Sato J, Masuda Y, Tsuchiya T., Kuramoto M., Elias P M, Feingold K R. Exposure to a dry environment enhances epidermal permeability barrier function. J Invest Dermatol 111 (1998) 858-863

Denda M. and Tsuchiya T. Barrier recovery rate varies time-dependently in human skin. Br J Dermatol 142:881-884, 2000

Elias P M and Feingold K R. Coordinate regulation of epidermal differentiation and barrier homeostasis. Skin Pharmacol Appl Skin Physiol 2001, 14:S28-S34

Grubauer G., Elias P M., Feingold K R. Transepidermal water loss: the signal for recovery of barrier structure and function. J Lipid Res 30:323-333

Inoue K, Koizumi S, Fuziwara S, Denda S, Inoue K, Denda M, Functional vanilloid receptors in cultured normal human keratinocytes. Biochem Biophys Res Commun 291:124-129, 2002

Moquich A., Hwang S W., Earley T J., Petrus M J., Murray A N., Spencer K S R., Andehazy M., Story G M., Patapoutian A. Impaired thermosensation in mice lacking TRPV3, a heat and camphor sensor in the skin. Science 307:1468-1472,2005

National Research Council (NRC) Guide. National Research Council, Washington, National Academy Press, 1996

Peier A M., Reeve A J., Anderson D A, Moqrich A., Earley T J., Hergarden A C., Story G M., Colley S., Hogenesch J B., McIntyre P., Bevan S., Patapotian A. A heat-sensitive TRP channel expressed in keratinocytes. Science 2002 296:2046-2049

Watanabe H., Davis J B., Smart D., Jermann J C., Smith G D., Hayes P., Vriens J., Cairns W., Wissenbach U., Prenen J., Flockerzi V., Droogmanns G., Benham C D., Nilius B. Activation of TRPV4 channels (hVRL-2/mTRP12) by phorbol derivatives. J Biol Chem 277: 13569-13577

What is claimed is:

1. A method for accelerating barrier recovery of skin by activating the TRPV4 on epidermal cell.

2. A method for accelerating barrier recovery of skin by blocking the TRPV1 on epidermal cell.

3. A method for accelerating barrier recovery of skin by activating the TRPV4 on epidermal cell, while blocking the TRPV1 on epidermal cell.
